(19) United States
(12) Patent Application Publication
(10) Pub. No.: US 2022/0195178 A1
(43) Pub. Date: Jun. 23, 2022

(54) BIORENEWABLE ELASTOMER GEL AND USES THEREOF

(71) Applicant: Momentive Performance Materials Inc., Waterford, NY (US)

(72) Inventors: Xu QIN, Waterford, NY (US); Benjamin FALK, Yorktown Heights, NY (US); Mana TAMAMI, Waterford, NY (US); Yuriy GALABURA, Waterford, NY (US); Amar PAWAR, Waterford, NY (US)

(21) Appl. No.: 17/560,021

(22) Filed: Dec. 22, 2021

Related U.S. Application Data
(60) Provisional application No. 63/130,218, filed on Dec. 23, 2020.

(51) Int. Cl. C08L 67/02; C08G 63/81; C08G 63/16; A61K 8/85; A61K 8/04

(52) U.S. Cl. CPC C08L 67/02; C08G 63/81; C08L 2312/00; A61K 8/85; A61K 8/042; C08G 63/16

(57) ABSTRACT

The present disclosure provides elastomer compositions and methods of preparing such compositions. The crosslinked polyester elastomer compositions can be formulated into various personal care formulations.

### FIELD

The present disclosure provides elastomer compositions and methods of preparing such compositions. Further, these elastomers can be converted to elastomer gels. These elastomers and elastomer gels can be biodegradable and produced from biorenewable raw materials. Further, these elastomer gels provide advantageous properties when combined with various personal care products.

### BACKGROUND

The personal care industry thrives on being able to deliver multiple performance products based on mixtures of several components, with each having performance characteristics important to or desirable in the final formulation. Silicone gels are commonly added in a variety of personal care formulations to enhance their aesthetics with respect to sensory, texture, rheology and optical performance. See for example, U.S. Pat. Nos. 4,987,169; 5,654,362; 5,760,116; 6,423,322; and 5,811,487.

However, traditional silicone gels have limited versatility in terms of compatibility with many natural oil, ester oil and other biobased ingredients which have growing interest in personal care formulation. Moreover, silicone polymers are difficult to degrade, biologically or otherwise. Particular silicone compounds used in various personal care products, cyclic siloxanes D4 (octamethylcyclotetrasiloxane) and D5 (decamethylcyclopentasiloxane), are air and water pollutants and show negative health effects on test animals. Therefore, biodegradable natural polymers gel made from biorenewable raw material with no health and environmental concern is highly desirable.

Polyesters are a class of compounds that contain ester functional group in their polymer chain. The ester group can be hydrolyzed when treated with certain biological catalysts or certain mixed cultures of microorganisms which make a large number of polyesters biodegradable. There is significantly growing interest in recent years to design and develop biobased polyesters from renewable resources as emollient, emulsifier, film former or other functional ingredient for personal care formulations. See for example, U.S. Pat. Nos. 8,414,906; 9,334,358; 6,540,987; 7,820,758. However, there is no polyester elastomer or gel reported yet to provide multi benefit to consumers as supplementary to silicone gel.

### SUMMARY

In a first aspect, the present disclosure provides an elastomer composition comprising the reaction product of: (i) at least one polycarboxylic acid, at least one polycarboxylic acid ester, or a combination thereof; and (ii) at least one polyol; wherein (a) the at least one polycarboxylic acid or at least one polycarboxylic acid ester and the at least one polyol have a total of at least five carboxyl and hydroxyl functional groups and (b) there must be at least three carboxyl or hydroxyl functional groups on the at least one polycarboxylic acid, the at least one polycarboxylic acid ester, or the at least one polyol.

In another aspect, the present disclosure provides an elastomer prepared by: (i) reacting: (a) at least one polycarboxylic acid, at least one polycarboxylic acid ester, or a combination thereof with (b) at least one polyol; in the presence of a first low molecular weight organic solvent thereby forming a crosslinking polymer structure; wherein (a) the at least one polycarboxylic acid, the at least one polycarboxylic acid ester, and the at least one polyol have a total of at least five carboxyl and hydroxyl functional groups and (b) there must be at least three carboxyl or hydroxyl functional groups on the at least one polycarboxylic acid, the at least one polycarboxylic acid ester, or the at least one polyol.

In an additional aspect, the present disclosure provides a method of preparing an elastomer comprising reacting (i) at least one polycarboxylic acid, at least one polycarboxylic acid ester, or a combination thereof and (ii) at least one polyol in a first solvent thereby forming a crosslinking polymer structure.

In a further aspect, the present disclosure provides for use of a gel prepared from an elastomer described here for a personal care formulation.

### DETAILED DESCRIPTION

### I. Definitions

Unless otherwise indicated, any atom with unsatisfied valences is assumed to have hydrogen atoms sufficient to satisfy the valences.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the following definitions shall apply unless otherwise indicated. For purposes of the present disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry,” Thomas Sorrell, University Science Books, Sausalito: 1999, and “March's Advanced Organic Chemistry,” 6^{th} Ed., Smith, M. B. and March, J., eds. John Wiley & Sons, New York: 2007, the entire contents of which are hereby incorporated by reference.

The term “alkyl”, as used herein by itself or as part of a group, refers to a straight- or branched-chain aliphatic hydrocarbon containing one to two hundred carbon atoms, i.e., a C₂-C₂₀₀ alkyl, or the number of carbon atoms designated, e.g., a C₁ alkyl such as methyl, a C₂ alkyl such as ethyl, etc. In one embodiment, the alkyl is a C₂-C₂₀₀ alkyl group. In another embodiment, the alkyl is a C₆-C₆₀ alkyl group. In another embodiment, the alkyl is a C₂-C₆₀ alkyl group. In another embodiment, the alkyl is a C₅-C₂₂ alkyl group. Examples of alkyl group include butyl, octyl, decyl, lauryl, cetyl (palmityl), and stearyl.

The term “alkene”, as used herein by itself or as part of a group, refers to an alkyl group containing one, two, three, or more carbon-to-carbon double bonds. In one embodiment, the alkene group is a C₂-C₂₀₀ alkylene group. In another embodiment, the alkene group is a C₆-C₆₀ alkene group. In another embodiment, the alkene group is a C₆-C₆₀ alkene group. In another embodiment, the alkene group is a C₂-C₆₀ alkene group. In another embodiment, the alkene group is a C₅-C₂₂ alkene group.

The term “alkyne”, as used herein by itself or as part of a group, refers to an alkyl group containing one, two, three, or more carbon-to-carbon triple bonds. In another embodiment, the alkyne is a C₂-C₂₀₀ alkyne group.

The term “cyclic”, as used herein by itself or as part of a group, refers to a stable cyclic compound containing three or more atoms. In an embodiment, the cyclic is a C₃-C₂₀₀ cyclic group. In an embodiment, the cyclic is a C₆-C₆₀ cyclic group. In an embodiment, the cyclic is a C₅-C₂₂ cyclic group. Examples of cyclic compound include benzene, cyclopentane, and cyclohexane.

The term “heteroalkyl”, as used herein by itself or as part of a group, refers to a stable straight or branched chain alkyl radical containing two to two hundred carbon atoms and at least one heteroatoms, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkyl is a C₆-C₆₀ heteroalkyl group. In an embodiment, the heteroalkyl is a C₂-C₆₀ heteroalkyl group. Examples of heteroalkyl compound include succinyl, adipoyl, and sebacoyl.

The term “heteroalkene”, as used herein by itself or as part of a group, refers to a stable straight or branched chain alkene radical containing two to two hundred carbon atoms and at least one heteroatoms, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkene is a C₆-C₆₀ heteroalkene group. In an embodiment, the heteroalkene is a C₂-C₆₀ heteroalkene group. Examples of heteroalkene compound include oleoyl, ricinolyl, and linoleoyl.

The term “heteroalkyne”, as used herein by itself or as part of a group, refers to a stable straight or branched chain alkyne radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. The term “heterocyclic”, as used herein by itself or as part of a group, refers to to a stable cyclic compound containing three or more atoms and at least one heteroatom, which can be the same or different, selected from O, N, or S. In an embodiment, the heterocyclic is a C₂-C₂₀₀ heterocyclic group. In an embodiment, the heterocyclic is C₆-C₆₀ heterocyclic group.

The term “heterocyclic”, as used herein by itself or as part of a group, refers to a stable cyclic compound containing two or more carbons atoms and at least one heteroatom, which can be the same or different, selected from O, N, or S, wherein the sulfur atom(s) can optionally be oxidized. In an embodiment, the heterocyclic is a C₂-C₂₀₀ heterocyclic group. In an embodiment, the heterocyclic is a C₆-C₆₀ heterocyclic group. In an embodiment, the heterocyclic is a C₅-C₂₂ heterocyclic group. Examples of heterocyclic compound include furan, oxolane, and thiophene.

Various aspect of the disclosure are described in greater detail below.

### II. Compositions

In one aspect, the present disclosure is directed to a crosslinked polyester elastomer composition comprising the reaction product of:
(i) at least one polycarboxylic acid, at least one polycarboxylic acid ester, or a combination thereof; and
(ii) at least one polyol;
wherein (a) the at least one polycarboxylic acid, the at least one polycarboxylic acid ester, and the at least one polyol have a total of at least five carboxyl and hydroxyl functional groups and (b) there must be at least three carboxyl or hydroxyl functional groups on the at least one polycarboxylic acid, the at least one polycarboxylic acid ester, or the at least one polyol.

### A. Components

a. Polycarboxylic Acid

In some aspects, the at least one polycarboxylic acid may be a compound of formula (I)

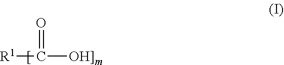

wherein

R¹ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and

m is an integer from 2 to 10.

In a further aspect, the compound is formula (I), wherein R¹ is C₆-C₆₀ alkyl group, C₆-C₆₀ heteroalkyl group, C₆-C₆₀ alkene group, C₆-C₆₀ heteroalkene group, C₆-C₆₀ cyclic group, or C₆-C₆₀ heterocyclic group; and m is an integer from 2 to 10.

In an aspect, the compound is formula (I), wherein m is an integer from 2 to 6. In another aspect, the compound is formula (I), wherein m is 2, 3, 4, 5, or 6.

In an aspect, the polycarboxylic acid is a product of a compound of formula (I), and/or a compound of formula (III) with a compound of formula (V).

In an aspect, the at least one polycarboxylic acid may be selected from the group consisting of citric acid, isocitric acid, aconitic acid, propane-1,2,3-tricarboxylic acid, trimesic acid, carballylic acid, C₅₄ trimer acid, mellitic acid, and combinations thereof. In a further aspect, the at least one polycarboxylic acid may be selected from the group consisting of citric acid, C₅₄ trimer acid, and combinations thereof.

In an aspect, C₃₆ dimer acid is a dicarboxylic acid prepared by dimerizing unsaturated fatty acids from plant oil.

In an aspect, C₅₄ trimer acid is a polycarboxylic acid prepared by trimerizing unsaturated fatty acids from plant oil.

In some aspects, the unsaturated fatty acids are palmitoleic acid, oleic acid, elaidic acid, vaccenic acid, linoleic acid, linoelaidic acid, or linolenic acid.

In some aspects, the plant oils are soybean oil, safflower oil, linseed oil, corn oil, sunflower oil, olive oil, canola oil, sesame oil, cottonseed oil, palm oil, rapeseed oil, tung oil, peanut oil, or milkweed oil.

In an aspects, the at least one carboxylic acid may be a dicarboxylic acid. In an aspect, the dicarboxylic acid is a compound of formula (II)

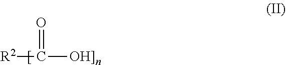

wherein

R² is C₂-C₂₀₀ alkyl group, C₂-C₂₀₀ heteroalkyl group, C₂-C₂₀₀ alkylene group, C₂-C₂₀₀ heteroalkylene group, C₂-C₂₀₀ alkyne group, C₂-C₂₀₀ heteroalkyne group, C₃-C₂₀₀ cyclic group, or C₂-C₂₀₀ heterocyclic group; and

n is an integer of 2 to 6.

In another aspect, the compound is formula (II), wherein R² is C₂-C₂₀₀ alkyl group, C₂-C₂₀₀ heteroalkyl group, C₂-C₂₀₀ alkene group, or C₂-C₂₀₀ heteroalkene group and n is 2. In a further aspect, the compound is of formula (II), wherein R² is C₂-C₆₀ alkyl group, C₂-C₆₀ heteroalkyl group, C₂-C₆₀ alkene group, or C₂-C₆₀ heteroalkene group and n is 2. In an aspect, the compound is of formula (II), wherein R² may be succinyl, adipoyl, sebacoyl, dilinoleyl, or trilinoleyl.

In an aspect, the compound is formula (II), wherein n may be 1 or 2.

In some aspects, the dicarboxylic acid may be selected from the group consisting of malonic acid, succinic acid, fumaric acid, glutaric acid, adipic acid, pimelic acid, suberic acid, azelaic acid, sebacic acid, undecanedioic acid, dodecanedioic acid, tridecanedioic acid, hexadecanedioic acid, C₂₁ dimer acid, C₃₆ dimer acid, hydrogenated C₃₆ dimer acid, aspartic acid, glutamic acid, tartaric acid, malic acid, and combinations thereof. In a further aspect, the dicarboxylic acid may be selected from the group consisting of malonic acid, succinic acid, adipic acid, azelaic acid, sebacic acid, undecanedioic acid, dodecanedioic acid, C₂₁ dimer acid, C₃₆ dimer acid, hydrogenated C₃₆ dimer acid, and combinations thereof. In one aspect, the dicarboxylic acid is selected from the group consisting of succinic acid, adipic acid, azelaic acid, sebacic acid, undecanedioic acid, dodecanedioic acid, C₂₁ dimer acid, C₃₆ dimer acid, hydrogenated C₃₆ dimer acid, and combinations thereof.

b. Polycarboxylic Acid Ester

In an aspect, the at least one polycarboxylic acid ester may be a compound of formula (III)

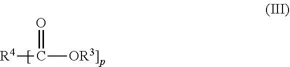

wherein

R³ is C₁-C₂₂ alkyl group, C₂-C₂₂ alkylene group, or C₃-C₂₂ cyclic group;

R⁴ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₂-C₂₀₀ cyclic group, or C₂-C₂₀₀ heterocyclic group; and

p is an integer from 3 to 10.

In another aspect, the compound is formula (III), wherein R³ is C₁-C₂₂ alkyl group or C₂-C₂₂ alkene group; R⁴ is C₂-C₂₀₀ alkyl group, C₂-C₂₀₀ heteroalkyl group, C₂-C₂₀₀ alkene group, or C₂-C₂₀₀ heteroalkene group; and p is an integer from 3 to 10.

In a further aspect, the compound is formula (III), wherein R³ is C₁-C₁₀ alkyl group; R⁴ is C₂-C₆₀ alkyl group, C₂-C₆₀ heteroalkyl group, C₂-C₆₀ alkene group, or C₂-C₆₀ heteroalkene group; and p is an integer from 3 to 10.

In an aspect, the compound is formula (III), wherein p is an integer from 3 to 6. In an aspect, the compound is formula (III), wherein p is 3, 4, 5, or 6.

In an aspect, the polycarboxylic acid ester is a product of a compound of formula (I), and/or a compound of formula (III) with a compound of formula (V).

In some aspects, the polycarboxylic acid ester may be selected from the group consisting of triethyl citrate, triethyl isocitrate, aconitic acid triethyl ester, propane-1,2,3-tricarboxylic acid triethyl ester, trimesic acid triethyl ester, carballylic acid triethyl ester, C₅₄ trimer acid triethyl ester, mellitic acid hexaethyl ester, and combinations thereof. In a further aspect, the polycarboxylic acid ester may be selected from the group consisting of triethyl citrate, C₅₄ trimer acid triethyl ester, and combinations thereof.

In an aspect, the at least one polycarboxylic acid ester may be a dicarboxylic acid ester. In some aspects, the dicarboxylic acid ester may be a compound of formula (IV)

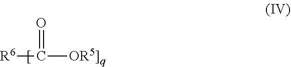

wherein

R⁵ is C₁-C₂₂ alkyl group, C₂-C₂₂ alkene group, or C₃-C₂₂ cyclic group;

R⁶ is C₂-C₂₀₀ alkyl group, C₂-C₂₀₀ heteroalkyl group, C₂-C₂₀₀ alkylene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and

q is 2.

In an aspect, the compound is formula (IV), wherein R⁵ is C₁-C₂₂ alkyl group or C₂-C₂₂ alkylene group; R⁶ is C₂-C₂₀₀ alkyl group, C₂-C₂₀₀ heteroalkyl group, C₂-C₂₀₀ alkene group, or C₂-C₂₀₀ heteroalkene group; and q is 2. In a further aspect, the compound is formula (IV), wherein R⁵ is C₁-C₁₀ alkyl group; R⁶ is C₂-C₆₀ alkyl group, C₂-C₆₀ heteroalkyl group, C₂-C₆₀ alkene group, or C₂-C₆₀ heteroalkene group; and q is 2.

In some aspects, the dicarboxylic acid may be selected from the group consisting of diethyl malonate, diethyl succinate, diethyl fumarate, diethyl adipate, diethyl pimelate, diethyl suberate, diethyl azelate, diethyl sebacate, diethyl undecanedioate, diethyl dodecanedioate, diethyl tridecanedioate, diethyl hexadecanedioiate, C₂₁ dimer acid diethyl ester, C₃₆ dimer acid diethyl ester, hydrogenated C₃₆ dimer acid diethyl ester, diethyl aspartate, diethyl glutamate, diethyl tartrate, diethyl malate, and combinations thereof. In a further aspect, the dicarboxylic acid ester may be selected from the group consisting of diethyl malonate, diethyl succinate, diethyl adipate, diethyl pimelate, diethyl azelate, diethyl sebacate, diethyl undecanedioate, C₂₁ dimer acid diethyl ester, C₃₆ dimer acid diethyl ester, hydrogenated C₃₆ dimer acid diethyl ester, and combinations thereof.

c. Polyol

In an aspect, the at least one polyol is a compound of formula (V)

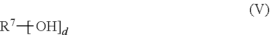

wherein

R⁷ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and

d is an integer from 2 to 10.

In an aspect, the compound is formula (V), wherein R⁷ is C₂-C₂₀₀ alkyl group, C₂-C₂₀₀ heteroalkyl group, C₂-C₂₀₀ alkene group, or C₂-C₂₀₀ heteroalkene group; and d is an integer from 2 to 10. In a further aspect, the compound is formula (V), wherein R⁷ is C₂-C₆₀ alkyl group, C₂-C₆₀ heteroalkyl group, C₂-C₆₀ alkene group, or C₂-C₆₀ heteroalkene group; and d is an integer from 2 to 10.

In an aspect, the compound is formula (V), wherein d is an integer from 2 to 6. In an aspect, the compound is formula (V), wherein d is 2, 3, 4, 5, or 6.

In an aspect, the polyol is a product of a compound of formula (I), and/or a compound of formula (III) with a compound of formula (V).

In an aspect, the C₃₆ dimer diol is the diol produced from a C₃₆ dimer acid.

In some aspects, the polyol may be selected from the group consisting of glycerol, diglycerol, polyglycerol, sorbitan, castor oil, hydrogenated castor oil, sugar alcohol, monosaccharide, disaccharides, oligosaccharide, polysaccharides, tannin, gallic acid, gluconic acid, lactobionic acid, gluconolactone, and combinations thereof. In a further aspect, the polyol may be selected from the group consisting of glycerol, diglycerol, polyglycerol, castor oil, hydrogenated castor oil, sorbitol, gallic acid, and combinations thereof. In another aspect, the alcohol may be selected from the group consisting of glycerol, diglycerol, polyglycerol, castor oil, hydrogenated castor oil, sorbitol, and combinations thereof.

In some aspects, the at least one polyol is a diol. In some aspects, the diol is a compound of formula (VI)

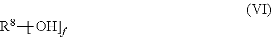

wherein

R⁸ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and

f is 2.

In an aspect, the compound is formula (VI), wherein R⁸ is C₂-C₂₀₀ alkyl group, C₂-C₂₀₀ heteroalkyl group, C₂-C₂₀₀ alkene group, or C₂-C₂₀₀ heteroalkene group; and f is 2. In a further aspect, the compound is formula (VI), wherein R⁸ is C₂-C₆₀ alkyl group, C₂-C₆₀ heteroalkyl group, C₂-C₆₀ alkene group, or C₂-C₆₀ heteroalkene group; and f is 2.

In some aspects, the diol may be selected from the group consisting of ethyleneglycol, 1,2-propanediol, 1,3-propanediol, 1,3-butanediol, 1,4-butanediol, 1,2-pentanediol, 1,3-pentanediol, 1,4-pentanediol, 1,5-pentanediol, 1,2-hexanediol, 1,5-hexanediol, 1,6-hexanediol, C₃₆ dimer diol, hydrogenated C₃₆ dimer diol, and combinations thereof. In a further aspect, the diol may be selected from the group consisting of 1,3-propanediol, 1,3-butanediol, 1,4-butanediol, 1,5-pentanediol, 1,6-hexanediol, C₃₆ dimer diol, and combinations thereof. In one aspect, the diol may be selected from the group consisting of 1,3-propanediol, 1,3-butanediol, 1,4-butanediol, 1,5-pentanediol, 1,6-hexanediol, C₃₆ dimer diol, hydrogenated C₃₆ dimer diol, and combinations thereof.

### A. Additional Components

In a further aspect, the composition may further comprise reacting the (i) at least one polycarboxylic acid, at least one polycarboxylic acid ester, or a combination thereof and (ii) the at least one polyol with at least one compound of formula (VII)

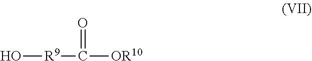

wherein

R⁹ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and

R¹⁰ is H, C₁-C₂₂ alkyl group, C₂-C₂₂ alkene group, or C₃-C₂₂ cyclic group.

In an aspect, the compound is formula (VII), wherein R⁹ is C₂-C₂₀₀ alkyl group, C₂-C₂₀₀ heteroalkyl group, C₂-C₂₀₀ alkene group, or C₂-C₂₀₀ heteroalkene group; and R¹⁰ is H, C₁-C₂₂ alkyl group, C₂-C₂₂ alkene group, or C₃-C₂₂ cyclic group. In a further aspect, the compound is formula (VII), wherein R⁹ is C₂-C₆₀ alkyl group, C₂-C₆₀ heteroalkyl group, C₂-C₆₀ alkene group, or C₂-C₆₀ heteroalkene group; and R¹⁰ is H, C₁-C₂₂ alkyl group, C₂-C₂₂ alkene group, or C₃-C₂₂ cyclic group.

In some aspects, the compound of formula (VII) may be selected from the group consisting of glycolic acid, lactic acid, salicylic acid, tropic acid, ricinoleic acid, isoricinoleic acid, lesquerolic acid, densipolic acid, auricolic acid, dimorphecolic acid, hydroxy palmitoleic acid, hydroxy palmitic acid, hydroxy oleic acid, 2-hydroxy stearic acid, 12-hydroxy stearic acid, and combinations thereof. In a further aspect, the compound of formula (VII) may be selected from the group consisting of ricinoleic acid, 12-hydroxy stearic acid, and combinations thereof.

a. Ratio

In some aspects, the ratio of the carboxylic functional group (—COOH) and the carboxyl ester functional group (—RCOOR—) to the hydroxyl functional group (—OH) in the reaction may range from about 1.5:1 to about 1:1.5. In other aspects, the ratio of the carboxylic functional group (—COOH) and the carboxyl ester functional group (—RCOOR—) to the hydroxyl functional group (—OH) in the reaction may range from about 1.25:1 to about 1:1.25. In further aspects, the ratio of the carboxylic functional group (—COOH) and the carboxyl ester functional group (—RCOOR—) to the hydroxyl functional group (—OH) in the reaction may be about 1.5:1, about 1.45:1.05, about 1.4:1.1, about 1.35:1.15, about 1.3:1.2, about 1.25:1.25, about 1.2:1.3, about 1.15:1.35, about 1.1:1.4, about 1.05:1.45, or about 1:1.5.

In another aspect, the ratio of the carboxylic functional group (—COOH) and the carboxyl ester functional group (—RCOOR—) to the hydroxyl functional group (—OH) in the reaction may range from about 1.2:1 to about 1:1.2. In a further aspect, the ratio of the carboxylic functional group (—COOH) and the carboxyl ester functional group (—RCOOR—) to the hydroxyl functional group (—OH) in the reaction may be about 1.2:1, about 1.15:1, about 1.1:1, about 1.05:1, about 1:1, about 1:1.05, about 1:1.1, about 1:1.15, about 1:1.2.

### B. Gel or Paste

In an aspect, the elastomer may be crumbled to form a crosslinked polyester elastomer powder.

In an aspect, a composition may be prepared by shearing the elastomer with a solvent, as described herein, to form a sheared gel. In another aspect, a composition may be prepared by combining the elastomer, as described herein, with a solvent thereby forming a mixture and shearing the mixture. In an aspect, the composition is a gel or a paste. In a further aspect, the composition is a gel.

In some aspects, the solvent will be as discussed below.

In some aspects, the viscosity of the gel may be from about 10 cp to about 1,000,000 cp as measured by rheometer at a shear rate of 0.1 s⁻¹. In another aspect, the viscosity of the gel may be from about 30,000 cp to about 500,000 cp. In some aspects, the viscosity of the gel may be about 10 cp, about 1,000 cp, about 5,000 cp, about 10,000 cp, about 15,000 cp, about 20,000 cp, about 25,000 cp, about 30,000 cp, about 35,000 cp, about 40,000 cp, about 45,000 cp, about 50,000 cp, about 55,000 cp, about 60,000 cp, about 65,000 cp, about 70,000 cp, about 75,000 cp, about 80,000 cp, about 85,000 cp, about 90,000 cp, about 95,000 cp, about 100,000 cp, about 150,000 cp, about 200,000 cp, about 250,000 cp, about 300,000 cp, about 350,000 cp, about 400,000 cp, about 450,000 cp, about 500,000 cp, about 550,000 cp, about 600,000 cp, about 650,000 cp, about 700,000 cp, about 750,000 cp, about 800,000 cp, about 850,000 cp, about 900,000 cp, about 950,000 cp, or about 1,000,000 cp.

In some aspects, the gel is comprised of particles of size from about 1 μm to about 500 μm as measured by laser diffraction particle size analyzer. In another aspect, the gel is comprised of particles of size from about 25 μm to about 400 μm. In some aspects, the gel is comprised of particles of size of about 1 μm, about 5 μm, about 10 μm, about 15 μm, about 20 μm, about 25 μm, about 30 μm, about 35 μm, about 40 μm, about 45 μm, about 50 μm, about 75 μm, about 100 μm, about 125 μm, about 150 μm, about 175 μm, about 200 μm, about 225 μm, about 250 μm, about 275 μm, about 300 μm, about 325 μm, about 350 μm, about 375 μm, or about 400 μm.

In an aspect, the elastomer may be prepared using the methods described herein.

### III. Methods

### A. Method of Preparing an Elastomer

a. Esterification Reaction

In an aspect, the present disclosure is directed to a method of preparing an elastomer comprising reacting (i) at least one polycarboxylic acid, at least one polycarboxylic acid ester, or a combination thereof and (ii) at least one polyol in a first solvent thereby forming a crosslinking polymer structure.

In some aspects, the method of preparing the elastomer, as described herein, is produced in an environmentally friendly process. In additional aspects, the method of preparing the elastomer, as described herein, utilizes no toxic raw materials. In further aspects, the method of preparing the elastomer, as described herein, generates no toxic side products.

In some aspects, the preparation of elastomer is under nitrogen protection, with vaccum, and combinations thereof.

In a further aspect, the preparation of elastomer is with vaccum in the range 100 Pa to 20,000 Pa.

b. Water Removal

In an aspect, the method further comprises removing water from the reaction. In a further aspect, the water is removed from the reaction by mixing and heating the reaction. In an aspect, the reaction may be heated to above about 120° C. In some aspects, the water is removed from the reaction by nitrogen flow or by vaccum.

Other methods for removing water from an esterification reaction are generally known to those of skill in the art.

c. Catalyst

In an aspect, the composition may further comprise a catalyst. Such catalysts include, without limit, methanesulfonic acid, p-toluenesulfonic acid, benzene sulfonic acid, sulfuric acid, amidosulfonic acid, sulfamic acid, sodium bisulfate, phosphoric acid, hydrochloric acid, hydrobromic acid, nitric acid, sodium hydroxide, potassium hydroxide, sodium methoxide, sodium ethoxide, potassium methoxide, potassium ethoxide, bismuth neodecanoate, bismuth(III) citrate, bismuth(III) chloride, bismuth(III) acetate, bismuth(III) phosphate, tin chloride, tin-pyrone, dibutyltin dilaurate, di-nbutyl-oxo-stannane, butyl stannoic acid, zinc chloride, zinc bromide, zinc carboxylic salt, zinc oxide, zinc hydroxy nitrate salt, zinc hydroxy acetate, triethylamine, tripropylamine, cocamidopropyl dimethylamine, stearamidopropyl dimethylamine, isostearamidopropyl dimethylamine, and combinations thereof. In still a further aspect, the catalyst is p-toluenesulfonic acid, methanesulfonic acid, phosphoric acid, bismuth neodecanoate, and combinations thereof. In a further aspect, the catalyst may be methanesulfonic acid.

In an additional aspect, the process further comprises (a) combining the crosslinking polymer structure with a second solvent thereby forming a swollen crosslinking polymer structure and (b) subjecting the swollen crosslinking polymer structure to shear force thereby forming a uniform polyester elastomer.

In some aspects, the shear force may be provided by any type of mixing and shearing equipment. In a further aspect, the mixing and shearing equipment may be batch mixer, planetary mixer, single or multiple screw extruder, dynamic or static mixer, colloid mill, homogenizer, sonolator, or a combination thereof.

d. Components

i. Polycarboxylic Acid

In some aspects, the at least one polycarboxylic acid may be compound of formula (I) described above.

In an aspects, the at least one carboxylic acid may be a dicarboxylic acid. In an aspect, the dicarboxylic acid is a compound of formula (II) described above.

ii. Polycarboxylic Acid Ester

In an aspect, the at least one polycarboxylic acid ester may be a compound of formula (III) described above. In an aspect, the at least one polycarboxylic acid ester may be a dicarboxylic acid ester. In some aspects, the dicarboxylic acid ester may be a compound of formula (IV) described above.

iii. Polyol

In an aspect, the at least one polyol is a compound of formula (V) described above.

In some aspects, the at least one polyol is a diol. In some aspects, the diol is a compound of formula (VI) described above.

iv. Additional Components

In a further aspect, the composition may further comprise reacting the (i) at least one polycarboxylic acid, at least one polycarboxylic acid ester, or a combination thereof and (ii) the at least one polyol with at least one compound of formula (VII) described above.

v. Solvent

In an aspect, the reaction may occur in the presence of a solvent. In some aspects, the solvent may be biobased or naturally derived. In an aspect, the solvent may be a triglyceride solvent, a mono-ester solvent, a di-ester solvent, a citrate ester solvent, an ether solvent, a carbonate solvent, a hydrocarbon solvent, a silicone solvent, and combinations thereof.

In an aspect, the solvent may be a triglyceride. In an aspect, the triglyceride solvent may be a compound of formula (VIII)

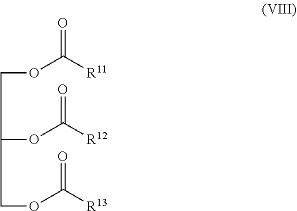

wherein
R¹¹, R¹², and R¹³ are independently C₁-C₃₅ alkyl group, C₁-C₃₅ heteroalkyl group, C₁-C₃₅ alkene group, and C₁-C₃₅ heteroalkene group.

In an aspect, the compound is formula (VIII), wherein R¹¹, R¹², and R¹³ are independently C₂-C₁₇ alkyl group or C₂-C₁₇ alkylene group.

In an aspect, the triglyceride solvent may be selected from the group consisting of caprylic/capric triglyceride, triheptanoin, corn oil, soybean oil, olive oil, rape seed oil, cotton seed oil, coconut oil, almond oil, argon oil, rosehip oil, black seed oil, grape seed oil, avocado oil, apricot kernel oil, geranium oil, lavender oil, rosehip oil, macadamia oil, eucalyptus oil, sardine oil, herring oil, safflower oil, linseed oil, sunflower oil, olive oil, canola oil, sesame oil, cottonseed oil, palm oil, rapeseed oil, tung oil, fish oil, peanut oil, cuphea oil, milkweed oil, salicornia oil, whale oil, castor oil, and combinations thereof. In a further aspect, the triglyceride solvent may be selected from the group consisting of caprylic/capric triglyceride, triheptanoin, and combinations thereof.

In an aspect, the solvent may be a mono-ester. In an aspect, the mono-ester solvent may be a compound of formula (IX)

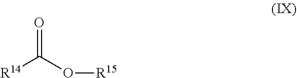

wherein
R¹⁴ is C₁-C₃₅ alkyl group, C₁-C₃₅ heteroalkyl group, C₂-C₃₅ alkene group, or C₂-C₃₅ heteroalkene group; and
R¹⁵ is H, C₁-C₃₅ alkyl group, C₁-C₃₅ heteroalkyl group, C₂-C₃₅ alkene group, or C₂-C₃₅ heteroalkylene group.

In an aspect, the compound is formula (IX), wherein R¹⁴ is C₅-C₁₇ alkyl group or C₅-C₁₇ alkene group and R¹⁵ is C₂-C₁₇ alkyl group or C₂-C₁₇ alkene group.

In an aspect, the mono-ester solvent is selected from the group consisting of coco-caprylate/caprate, coco-caprylate, coco-caprate, jojoba oil, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octydodecyl erucate, C₁₂-C₁₃ alkyl lactate, C₁₂-C₁₅ alkyl lactate, isostearyl lactate, glycereth-5 lactate, lauryl lactate, myristyl lactate, oleyl lactate, laureth-2 benzoate, C₁₂-C₁₅ alkyl benzoate, C₁₂-C₁₅ pareth-3 benzoate, dipropylene glycol benzoate, isodecyl salicylate, C₁₂-C₁₅ alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2 ethylhexanoate, cetearyl ethylhexanoate, isodecyl neopentanoate, isostearyl neopentanoate, nyristyl neopentanoate, isostearyl behenate, octyldodecyl neopentanoate, tridecyl neopentanoate, and combinations thereof.

In a further aspect, the mono-ester solvent is selected from the group consisting of coco-caprylate/caprate, coco-caprylate, jojoba oil, isoamyl laurate, methylheptyl isostearate, C₁₂-C₁₃ alkyl lactate, C₁₂-C₁₅ alkyl lactate, lauryl lactate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, and combinations thereof. In still a further aspect, the mono-ester solvent is selected from the group consisting of coco-caprylate/caprate, coco-caprylate, isoamyl laurate, isononyl isononanoate, heptyl undecylenate, jojoba oil, jojoba esters, and combinations thereof.

In an aspect, the solvent may be a di-ester solvent. In an aspect, the di-ester solvent may be a compound of formula (X), formula (XI), or formula (XII)

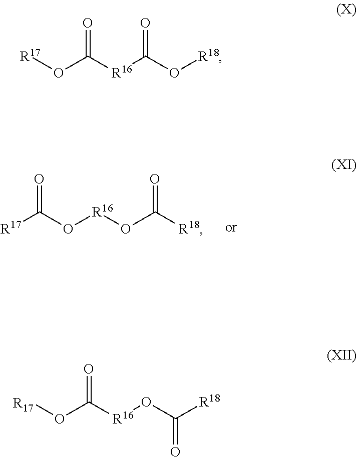

wherein
R¹⁶ is C₁-C₃₅ alkyl group, C₁-C₃₅ heteroalkyl group, C₂-C₃₅ alkene group, or C₂-C₃₅ heteroalkene group; and
R¹⁷ and R¹⁸ are independently H, C₁-C₃₅ alkyl group, C₁-C₃₅ heteroalkyl group, C₂-C₃₅ alkene group, or C₂-C₃₅ heteroalkene group.

In an aspect, the compound is formula (X), formula (XI), or formula (XII), wherein R¹⁶ is C₂-C₁₀ alkyl group or C₂-C₁₀ alkene group and R¹⁷ and R¹⁸ are independently C₁-C₁₂ alkyl group or C₂-C₁₂ alkene group.

In an aspect, the di-ester solvent may be selected from the group consisting of diethyl succinate, dibutyl succinate, diethyhexyl succinate, diisopropyl sebacate, dimethyl sebacate, diethyl sebacate, dibutyl sebacate, diisostearyl dimer, diisostearyl malate, isostearyl stearoyl stearate, isocetyl stearoyl stearate, octyldodecyl stearoyl stearate, diethylhexyl malate, diethylhexyl maleate, dipropylene glycol dibenzoate, dicapryl adipate, dicaprylyl maleate, diisopropyl dimer, diisopropyl adipate, diisobutyl adipate, diisopropyl sebacate, diisostearyl dimer, diethyhexyl succinate, diethylene glycol diethylhexanoate, neopentyl glycol dicaprate, propylene glycol dicaprylate/dicaprate, neopentyl glycol diisostearate, neopentyl glycol diethylhexanoate, neopentyl glycol diheptanoate, and combinations thereof. In a further aspect, the di-ester solvent may be selected from the group consisting of dicapryl adipate, dicaprylyl maleate, diisopropyl adipate, diisobutyl adipate, diethyl succinate, dibutyl succinate, diethyhexyl succinate, diisopropyl sebacate, dimethyl sebacate, diethyl sebacate, dibutyl sebacate, neopentyl glycol diethylhexanoate, neopentyl glycol diheptanoate, and combinations thereof.

In an aspect, the solvent may be a citrate ester. In an aspect, the citrate ester may be a compound of formula (XIII)

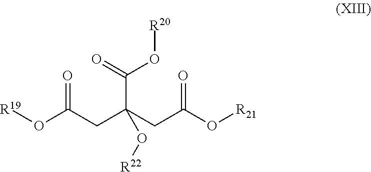

wherein
R¹⁹, R²⁰, R²¹, and R²² are independently H, C₁-C₃₅ alkyl group, C₁-C₃₅ heteroalkyl group, C₂-C₃₅ alkene group, or C₂-C₃₅ heteroalkene group.

In an aspect, the compound is formula (XIII), wherein R¹⁹, R²⁰, and R²¹ are independently C₁-C₁₀ alkyl group or C₂-C₁₀ alkene group and R²² is an acetyl group.

In an aspect, the citrate ester solvent may be a compound selected from the group consisting of tricaprylyl citrate, triisostearyl citrate, triisocetyl citrate, trioctyldodecyl citrate, triethyl citrate, tributyl citrate, acetyl triethyl citrate, acetyl tributyl citrate, trioctyldodecyl citrate, triisocetyl citrate, and combinations thereof.

In an aspect, the solvent may be an ether solvent. In an aspect, the ether solvent may be a compound of formula (XIV)

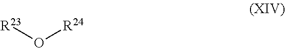

wherein R²³ and R²⁴ are independently H, C₂-C₂₀ alkyl group, C₂-C₂₀ heteroalkyl group, C₂-C₂₀ alkene group, or C₂-C₂₀ heteroalkene group.

In an aspect, the compound is formula (XIV), wherein R²³ and R²⁴ are independently C₂-C₂₀ alkyl group.

In an aspect, the ether solvent may be selected from the group consisting of dicaprylyl ether, didecyl ether, panthenyl ethyl ether, dicetyl ether, dimyristyl ether, distearyl ether, distearyl ether, dilauryl ether, and combinations thereof. In a further aspect, the ether solvent may be selected from the group consisting of dicaprylyl ether, didecyl ether, and combinations thereof.

In an aspect, the solvent may be a carbonate solvent. In an aspect, the carbonate solvent may be a compound of formula (XV)

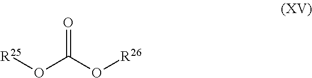

wherein
R²⁵ and R²⁶ are independently H, C₂-C₂₀ alkyl group, C₂-C₂₀ heteroalkyl group, C₂-C₂₀ alkene group, or C₂-C₂₀ heteroalkene group.

In an aspect, the compound is formula (XV), wherein R²⁵ and R²⁶ are independently C₂-C₂₀ alkyl group.

In an aspect, the carbonate solvent may be selected from the group consisting of dicaprylyl carbonate, diethylhexyl carbonate, and combinations thereof.

In an aspect, the hydrocarbon solvent is selected from the group consisting of farnesene, hydrogenated farnesene, coconut alkanes, coconut/palm kernel alkanes, C₉-C₁₂ alkane, C₁₀-C₁₃ alkane, C₁₂-C₁₇ alkane, C₁₃-C₁₄ alkane, C₁₃-C₁₅ alkane, C₁₄-C₁₇ alkane, C₁₄-C₁₉ alkane, C₁₄-C₂₀ alkane, C₁₄-C₂₂ alkane, C₁₅-C₁₉ alkane, C₂₁-C₂₈ alkane, C₁₇-C₂₃ alkane, C₉-C₁₂ isoalkane, C₉-C₁₃ isoalkane, C₉-C₁₄ isoalkane, C₉-C₁₆ isoalkane, C₁₀-C₁₁ isoalkane, C₁₀-C₁₂ isoalkane, C₁₀-C₁₃ isoalkane, C₁₁-C₁₂ isoalkane, C₁₁-C₁₃ isoalkane, isoalkane, C₁₂-C₁₄ isoalkane, C₁₂-C₁₅ isoalkane, C₁₂-C₂₀ isoalkane, C₁₃-C₁₄ isoalkane, C₁₃-C₁₆ isoalkane, C₁₄-C₁₆ isoalkane, C₁₅-C₁₉ isoalkane, diethylhexylcyclohexane, undecane, tridecane, tetradecane, pentadecane, hexadecane, octadecane, docosane, squalane, hydrogenated polyisobutene, polybutene, hydrogenated polydecene, hydrogenated didecene, mineral oil, liquidum, petrolatum, dodecane, isohexadecane, isododecane, isoeicosane, and combinations thereof. In a further aspect, hydrocarbon solvent is selected from the group consisting of farnesene, hydrogenated farnesene, coconut alkanes, C₉-C₁₂ alkane, C₁₃-C₁₅ alkane, C₁₄-C₁₉ alkane, C₁₄-C₂₀ alkane, C₁₄-C₂₂ alkane, C₁₅-C₁₉ alkane, C₁₃-C₁₆ isoalkane, dodecane, undecane, tridecane, tetradecane, pentadecane, hexadecane, octadecane, squalane, isododecane, isohexadecane, and combinations thereof. In another aspect, the hydrocarbon solvent is selected from the group consisting of hydrogenated farnesene, coconut alkanes, C₉-C₁₂ alkane, C₁₃-C₁₅ alkane, C₁₃-C₁₆ isoalkane, C₁₄-C₁₉ alkane, dodecane, tetradecane, isododecane, hexadecane, octadecane, and combinations thereof.

In an aspect, the solvent may be a silicone solvent. In some aspects, the silicone solvent may be selected from the group consisting of dimethicone, phenyl dimethicone, caprylyl methicone, ethyl trisiloxane, cyclotetrasiloxane, cyclopentasiloxane, cyclohexasiloxane, and combinations thereof.

In an aspect, the amount of the first solvent in the reaction may be present from about 10% to about 70% of the total weight. In an aspect, the amount of the first solvent in the reaction may be present from about 20% to about 70% of the total weight of the epoxidized vegetable oil, the crosslinker, and the solvent. In a further aspect, the amount of the first solvent present may be from about 30% to about 50% of the total weight of the carboxylic acid, the alcohol, and the solvent. In another aspect, the amount of the first solvent present may be about 10%, about 15%, about 20%, about 25%, about 30%, about 35%, about 40%, about 45%, about 50%, about 55%, about 60%, about 65%, or about 70% of the total weight.

vi. Temperature

In an aspect, the reaction may occur at a temperature from about 60° C. to about 250° C. In an aspect, the reaction may occur at a temperature from about 50° C. to about 225° C., about 75° C. to about 200° C., about 100° C. to about 195° C., about 125° C. to about 180° C., or about 120° C. to about 180° C. In a further aspect, the reaction may occur at a temperature from about 120° C. to about 180° C. In another aspect, the reaction may occur at a temperature of about 100° C., about 110° C., about 115° C., about 120° C., about 125° C., about 130° C., about 135° C., about 140° C., about 145° C., about 150° C., about 155° C., about 160° C., about 165° C., about 170° C., about 175° C., about 180° C., about 185° C., about 190° C., about 195° C., about 200° C., about 205° C., about 210° C., about 215° C., about 220° C., about 225° C., about 230° C., about 235° C., about 240° C., about 245° C., or about 250° C.

vii. Time

In an aspect, the reaction time may be from about 1 hour to about 72 hours. In a further aspect, the reaction time may be from about 6 hours to about 24 hours. In a further aspect, the reaction time may be from about 8 hours to about 27 hours. In another aspect, the reaction time may be about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, or about 27 hours.

e. Gel or Paste

In an aspect, a gel or paste from an elastomer described herein can be prepared by the methods described above. In an aspect, the uniform polyester elastomer may be crumbled to form an elastomer powder. In an aspect, the uniform polyester elastomer is processed into a gel or a paste. In a further aspect, the uniform polyester elastomer is a gel.

In an aspect, polyester elastomer is mixed with a solvent before processed to make a gel.

In an aspect, polyester elastomer is swelled in a solvent before processed to make a gel. In another aspect, the time polyester elastomer swelling in a solvent is from 1 hour to 1 week.

In an aspect, polyester elastomer and solvent mixture is processed by homogenizer to produce a gel.

In some aspects, the viscosity of the gel is described above. In some aspects, the gel is comprised of particles of size described above.

### IV. Personal Care Formulations

In an additional aspect of the present disclosure, the elastomers described herein may be incorporated into a personal care formulation. In some aspects, the gels prepared from the elastomers described herein may be incorporated into a personal care formulation.

In some aspects, the personal care formulation further may comprise a preservative, an antioxidant, a chelating agent, a gum or thickener, an oil, a wax, a fragrance, an essential oil, an emulsifier, a surfactant, and combinations thereof.

In an aspect, the personal care item is a personal care application that can be a deodorant, an antiperspirant, a skin cream, a facial cream, a hair shampoo, a hair conditioner, a mousse, a hair styling gel, a hair spray, a protective cream, a lipstick, a facial foundations, blushes, makeup, and mascara, a skin care lotion, a moisturizer, a facial treatment, a personal cleanser, a facial cleanser, a bath oil, a perfume, a shaving cream, a pre-shave lotion, an after-shave lotion, a cologne, a sachet, or a sunscreen.

### V. Additional Formulations

In other aspects, the products of the present disclosure, i.e., the crosslinked polyester elastomer, may be added to formulations comprising pharmaceuticals, biocides, herbicides, pesticides, or other biologically active substances.

In some aspects, the products of the present disclosure, i.e., the crosslinked polyester elastomer, may be used to incorporate water and water-soluble substances into hydrophobic systems. In other aspects, the products of the present disclosure, i.e., the crosslinked polyester elastomer, may modify the rheological, physical or energy absorbing properties of oil phases in either their neat or finished condition.

### VI. Use of the Gels

In one aspect, the disclosure relates to the use of a gel composition described herein for personal care formulations, such as a personal care application. The personal care applications can be any described above.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: Esterification to Prepare Polyester Elastomer

In a suitable vessel equipped with agitation, heat and an ability to distill off water, 110 g hydrogenated dimer acid was added along with 140 g hydrogenated castor oil. Next 250 g hemisqualane was added as solvent. Finally 1.25 g methanesulfonic acid was added. After all ingredients have been charged under agitation, the temperature of the mass was raised to 140° C., and water was stripped off as formed. The temperature was held 4-8 hours or until gelation took, place and polymer elastomer was formed. Thereafter the elastomer was broken into a powder by mechanical stirring.

### Example 2: Esterification to Prepare Polyester Elastomer

In a suitable vessel equipped with agitation, heat and an ability to distill off water, 50 g sebacic acid was added along with 200 g hydrogenated castor oil. Next 250 g hemisqualane was added as solvent. Finally 1.25 g of methanesulfonic acid was added. After all ingredients have been charged under agitation, the temperature of the mass was raised to 140° C., and water was stripped off as formed. The temperature was held 4-8 hours or until gelation took, place and polymer elastomer was formed. Thereafter the elastomer was broken into a powder by mechanical stirring.

### Example 3: Esterification to Prepare Polyester Elastomer

In a suitable vessel equipped with agitation, heat and an ability to distill off water, 227 g dimer acid was added along with 280 g hydrogenated castor oil. Next 500 g dicaprylyl ether was added as solvent. Finally 2 g methanesulfonic acid was added. After all ingredients have been charged under agitation, the temperature of the mass was raised to 140° C., and water was stripped off as formed. The temperature was held 16-24 hours or until gelation took, place and polymer elastomer was formed. Thereafter the elastomer was broken into a powder by mechanical stirring.

### Example 4: Esterification to Prepare Polyester Elastomer

In a suitable vessel equipped with agitation, heat and an ability to distill off water, 65 g dimer acid was added along with 80 g hydrogenated castor oil and 20 g 12-hydroxystearic acid. Next 200 g tetradecane was added as solvent. Finally 1 g of methanesulfonic acid was added. After all ingredients have been charged under agitation, the temperature of the mass was raised to 130° C., and water was stripped off as formed. The temperature was held 12-18 hours or until gelation took, place and polymer elastomer was formed. Thereafter the elastomer was broken into a powder by mechanical stirring.

### Example 5: Preparation of Polyester Gel from Polyester Elastomer

After a very nice elastomer was produced which did not separate upon standing, 380 g coco-caprylate/caprate was added into the vessel to mix with 120 g elastomer. After the gel and solvent mixture sit in vessel for 12 hours it was homogenized with an Gaulin homogenizer to produce a creamy, translucent gel of very smooth consistency, suitable for use in personal care formulations.

### Example 6: Preparation of a Natural Oil/Water Foundation

Components of a natural oil/water foundation are listed in Table 1.

| | INCI name | % |
|---|---|---|
| A | Aqua | Up to 100.0 |
| | Xanthan gum | 3.0 |
| | Glycerin | 3.0 |
| | Disodium EDTA | 0.05 |
| | Hydroxyethyl Acrylate/Sodium | 2.0 |
| | Acryloyldimethyl Taurate Copolymer & | |
| | Squalane & Polysorbate 60 | |
| B | PEG-20 Methyl Glucose Sesquistearate | 3.5 |
| | Methyl Glucose Sesquistearate | 2.0 |
| | Cl 77891 (and) Triethoxycaprylylsilane | 5.03 |
| | Cl 77492 (and) Triethoxycaprylylsilane | 0.67 |
| | Cl 77491 (and) Triethoxycaprylylsilane | 0.204 |
| | Cl 77499 (and) Triethoxycaprylylsilane | 0.096 |
| | Cetyl Alcohol | 1.5 |
| | C13-15 Alkane | 4.0 |
| | Ethylhexyl Stearate | 3.0 |
| | Coco-Caprylate/Caprate | 2.0 |
| | Isononyl Isononanoate | 4.0 |
| | Gel from example 5 | 5.0 |
| C | Preservative | q.s. |
| | Fragrance | q.s. |

Combine phase A ingredients and heat to 50° C. with stirring. Wet powders with solvent mixture in phase B and homogenize. Add the homogenized mixture to the rest of phase B and heat to 50° C. Add phases B into phase A and mix for 10 minutes until content is homogeneous. Add phase C and homogenize.

### Example 7: Preparation of a Natural Water/Oil Foundation

| | INCI name | % |
|---|---|---|
| A | Water | Up to 100 |
| | Glycerin | 3.0 |
| | Sodium Chloride | 1.0 |
| B | PEG-30 Dipolyhydroxystearate | 5.0 |
| | Polyglyceryl-3 Diisostearate | 2.0 |
| | Pentaerythrityl Distearate | 1.0 |
| | Dicaprylyl Carbonate (and) Stearalkonium | 6.0 |
| | Hectorite (and) Propylene Carbonate | |
| | Isononyl Isononanoate | 4.0 |
| | Coco-Caprylate | 4.0 |
| | Propylheptyl Caprylate | 7.0 |
| | Gel from example 5 | 5.0 |
| | CI 77891 (and) Triethoxycaprylylsilane | 4.19 |
| | CI 77492 (and) Triethoxycaprylylsilane | 0.56 |
| | CI 77491 (and) Triethoxycaprylylsilane | 0.17 |
| | CI 77499 (and) Triethoxycaprylylsilane | 0.08 |
| | Hordeum Vulgare Seed Flour | 2.0 |
| | Aluminum Starch Octenylsuccinate | 4.0 |
| | Silica | 1.0 |
| | Boron Nitride | 1.0 |
| C | Phenoxyethanol (and) Ethylhexylglycerin | 1.0 |

Mix phase A and heat to 50° C. Wet powders with solvents and homogenize until smooth. Add to the rest of phase B and heat to 50° C. Slowly add phase A to phase B while mixing. Homogenize phase A+B. Cool down and add phase ® C.

### Example 8: Preparation of a Matte Lipstick with Pigments

| Phase | INCI name | % |
|---|---|---|
| A | Ricinus Communis (Castor) Seed Oil | To 100 |
| | Titanium Dioxide (CI 77891)) | 2.00 |
| | CI 77499 | 0.04 |
| | CI 77492 | 0.40 |
| | CI 45410 | 0.50 |
| | Helianthus Annuus (Sunflower) Seed Oil | 18.00 |
| | Euphorbia Cerifera (Candelilla) Wax | 15.00 |
| | Butyrospermum Parkii (Shea) Butter | 5.00 |
| | Cocos Nucifera | 20.00 |
| | Gel from example 5 | 25.00 |
| | Neossance Hemisqualane | 5.00 |
| | Caprylyl Caprylate/Caprate | 20.00 |

Combine the phase (A) ingredients and heat to 70 C until homogenous. Pour into mold.

### Example 9: Preparation of a Natural Primer

| Phase | INCI Name | Wt (%) |
|---|---|---|
| A | Gel from example 5 | 70.0 |
| | Caprylic/Capric Triglyceride (and) | 5.0 |
| | Polyurethane-79 | |
| | Heptyl Undecylenate | 25 |

Mix all ingredients and heat to 80-90° C. using cowles mixer. Ensure the oil thickener is dissolved, then pour the mixture into the jar and let it cool overnight.

### Example 10: Preparation of a Moisturizing Skin Cream

| Phase | INCI Name | Wt (%) |
|---|---|---|
| A | Aqua | Up to 100.0 |
| | Xanthan Gum | 0.2 |
| | Glycerin | 0.5 |
| B | Disodium EDTA | 0.05 |
| | Glyceryl Stearate Citrate | 3.0 |
| | Sodium Stearoyl Glutamate | 0.5 |
| | Glyceryl Stearate | 1.5 |
| | Cetearyl Alcohol | 2.0 |
| | C13-15 Alkane | 5.0 |
| | PPG-15 Stearyl Ether | 5.0 |
| | Caprylic-Capric Triglyceride | 5.0 |
| | Ge from example 5 | 5.0 |
| C | Preservative | q.s. |
| | Fragrance | q.s. |

Mix all the ingredients of phase A, heat to 75° C. Mix all the ingredients of phase B, heat to 70° C. Add phase B to phase A with intensive stirring. Homogenize the mixture for 1-2 minutes. Cool to 50° C. under gentle stirring. Add preservative and fragrance as desired, and mix well.

### Example 11: Preparation of an Oil in Water Sunscreen

| Phase | INCI Name | Wt (%) |
|---|---|---|
| A | Water | Up to 100.0 |
| | Xanthan gum | 0.2 |
| | Glycerin | 0.5 |
| | Disodium EDTA | 0.1 |
| | Carbomer | 0.2 |
| | Butylene glycol | 2.0 |
| B | Sodium stearoyl glutamate | 1.0 |
| | Glyceryl stearate | 3.0 |
| | Ethylhexyl salicylate | 5.0 |
| | Homosalate | 15.0 |
| | Avobenzone | 3.0 |
| | Octocrylene | 10 |
| C | Gel from example 5 | 5.0 |
| D | Preservative | as needed |

Wet xanthan gum with glycerin and butylene glycol; add the rest of the ingredients of phase A and heat to 40° C. until homogenous. Mix ingredients of phase B and heat to 70-80° C. until homogenous. Add phase C to B and mix. Slowly add phase B+C to A. Homogenize the mixture 11,000 rpm for 1 minute. Cool down and add preservative.

### OTHER ASPECTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed.

What is claimed is:

1. An elastomer composition comprising the reaction product of:
(i) at least one polycarboxylic acid, at least one polycarboxylic acid ester, or a combination thereof; and
(ii) at least one polyol;
wherein (a) the at least one polycarboxylic acid or at least one polycarboxylic acid ester and the at least one polyol have a total of at least five carboxyl and hydroxyl functional groups and (b) there must be at least three carboxyl or hydroxyl functional groups on the at least one polycarboxylic acid, the at least one polycarboxylic acid ester, or the at least one polyol.

2. The elastomer of claim 1, wherein the at least one polycarboxylic acid is a compound of formula (I) 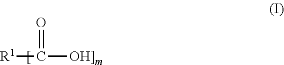
wherein
R¹ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
m is an integer from 2 to 10.

3. The elastomer of of claim 2, wherein the polycarboxylic acid is selected from the group consisting of citric acid, isocitric acid, aconitic acid, propane-1,2,3-tricarboxylic acid, trimesic acid, carballylic acid, C₅₄ trimer acid, mellitic acid, and combinations thereof.

4. The elastomer of claim 1, wherein the at least one polycarboxylic acid is a dicarboxylic acid that is a compound of formula (II) 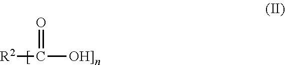
wherein
R² is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
n is 2.

5. The elastomer of claim 4, wherein the dicarboxylic acid is selected from the group consisting of succinic acid, adipic acid, azelaic acid, sebacic acid, dodecanedioic acid, hexadecanedioic acid, C₂₁ dimer acid, C₃₆ dimer acid, hydrogenated C₃₆ dimer acid, aspartic acid, glutamic acid, tartaric acid, malic acid, and combinations thereof.

6. The elastomer of claim 1, wherein the at least one polycarboxylic acid ester is a compound of formula (III) 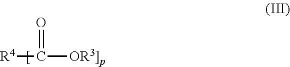
wherein
R³ is C₁-C₂₂ alkyl group, C₂-C₂₂ alkene group, or C₃-C₂₂ cyclic group;
R⁴ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
p is an integer from 3 to 10.

7. The elastomer of of claim 6, wherein the polycarboxylic acid ester is selected from the group consisting of triethyl citrate, triethyl isocitrate, aconitic acid triethyl ester, propane-1,2,3-tricarboxylic acid triethyl ester, trimesic acid triethyl ester, carballylic acid triethyl ester, C₅₄ trimer acid triethyl ester, mellitic acid hexaethyl ester, and combinations thereof.

8. The elastomer of claim 6, wherein the polycarboxylic acid ester is a dicarboxylic acid ester that is a compound of formula (IV) 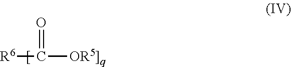
wherein
R⁵ is C₁-C₂₂ alkyl group, C₂-C₂₂ alkene group, or C₃-C₂₂ cyclic group;
R⁶ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
q is 2.

9. The elastomer of claim 8, wherein the dicarboxylic acid ester is selected from the group consisting of diethyl malonate, diethyl succinate, diethyl adipate, diethyl pimelate, diethyl azelate, diethyl sebacate, diethyl undecanedioate, C₂₁ dimer acid diethyl ester, C₃₆ dimer acid diethyl ester, hydrogenated C₃₆ dimer acid diethyl ester, and combinations thereof.

10. The elastomer of claim 1, wherein the at least one polyol is a compound of formula (V) 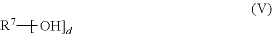
wherein
R⁷ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
d is an integer from 2 to 10.

11. The elastomer of claim 10, wherein the polyol is selected from the group consisting of glycerol, diglycerol, polyglycerol, sorbitol, castor oil, hydrogenated castor oil, sugar alcohol, monosaccharide, disaccharides, oligosaccharide, polysaccharides, tannin, gallic acid, gluconic acid, lactobionic acid, gluconolactone, and combinations thereof.

12. The elastomer of claim 10, wherein the polyol is a diol that is a compound of formula (VI) 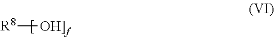
wherein
R⁸ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
f is 2.

13. The elastomer of claim 12, wherein the diol is selected from the group consisting of ethyleneglycol, 1,2-propanediol, 1,3-propanediol, 1,3-butanediol, 1,4-butanediol, 1,2-pentanediol, 1,3-pentanediol, 1,4-pentanediol, 1,5-pentanediol, 1,2-hexanediol, 1,5-hexanediol, 1,6-hexanediol, C₃₆ dimer diol, hydrogenated C₃₆ dimer diol, and combinations thereof.

14. The elastomer of claim 1, wherein the ratio of the carboxyl functional group (—COOH) and the carboxyl ester functional group (—RCOOR—) to the hydroxyl functional group (—OH) is from about 1.5:1 to about 1:1.5.

15. The elastomer of claim 1, wherein the elastomer is prepared by reacting:
i. at least one polycarboxylic acid, at least one polycarboxylic acid ester, or a combination thereof with
ii. at least one polyol;
in the presence of a first solvent thereby forming a crosslinking polymer structure;
wherein (a) the at least one polycarboxylic acid, the at least one polycarboxylic acid ester, and the at least one polyol have a total of at least five carboxyl and hydroxyl functional groups and (b) there must be at least three carboxyl or hydroxyl functional groups on the at least one polycarboxylic acid, the at least one polycarboxylic acid ester, or the at least one polyol.

16. The elastomer of claim 15, wherein the solvent is selected from the group consisting of a triglyceride solvent, a mono-ester solvent, a di-ester solvent, a citrate ester solvent, an ether solvent, a carbonate solvent, a hydrocarbon solvent, a silicone solvent, and combinations thereof.

17. The elastomer of claim 16, wherein the triglyceride solvent comprising the compound of formula (VIII) 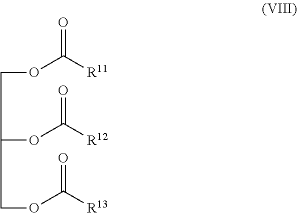
wherein
R¹¹, R¹², and R¹³ are independently C₁-C₃₅ alkyl group, C₁-C₃₅ heteroalkyl group, C₂-C₃₅ alkene group, or C₂-C₃₅ heteroalkene group.

18. The elastomer of claim 17, wherein the triglyceride solvent is selected from the group consisting of caprylic/capric triglyceride, triheptanoin, corn oil, soybean oil, olive oil, rape seed oil, cotton seed oil, coconut oil, almond oil, argon oil, rosehip oil, black seed oil, grape seed oil, avocado oil, apricot kernel oil, geranium oil, lavender oil, rosehip oil, macadamia oil, eucalyptus oil, sardine oil, herring oil, safflower oil, linseed oil, sunflower oil, olive oil, canola oil, sesame oil, cottonseed oil, palm oil, rapeseed oil, tung oil, fish oil, peanut oil, cuphea oil, milkweed oil, salicornia oil, whale oil, castor oil, and combinations thereof.

19. The elastomer of claim 16, wherein the mono-ester solvent is a compound of formula (IX) 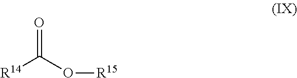
wherein
R¹⁴ is C₁-C₃₅ alkyl group, C₁-C₃₅ heteroalkyl group, C₂-C₃₅ alkene group, or C₂-C₃₅ heteroalkene group; and
R¹⁵ is H, C₁-C₃₅ alkyl group, C₁-C₃₅ heteroalkyl group, C₂-C₃₅ alkene group, or C₂-C₃₅ heteroalkene group.

20. The elastomer of claim 19, wherein the mono-ester solvent is selected from the group consisting of coco-caprylate/caprate, coco-caprylate, coco-caprate, jojoba oil, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octydodecyl erucate, C₁₂-C₁₃ alkyl lactate, C₁₂-C₁₅ alkyl lactate, isostearyl lactate, glycereth-5 lactate, lauryl lactate, myristyl lactate, oleyl lactate, laureth-2 benzoate, C₁₂-C₁₅ alkyl benzoate, C₁₂-C₁₅ pareth-3 benzoate, dipropylene glycol benzoate, isodecyl salicylate, C₁₂-C₁₅ alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2 ethylhexanoate, cetearyl ethylhexanoate, isodecyl neopentanoate, isostearyl neopentanoate, nyristyl neopentanoate, isostearyl behenate, octyldodecyl neopentanoate, tridecyl neopentanoate, and combinations thereof.

21. The elastomer of claim 16, wherein the di-ester solvent is a compound of formula (X), formula (XI), or formula (XII) 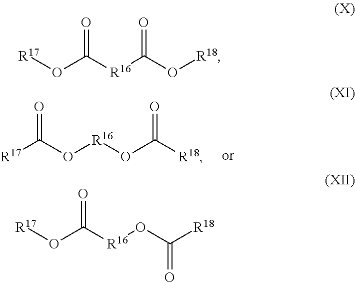
wherein
R¹⁶ is C₁-C₃₅ alkyl group, C₁-C₃₅ heteroalkyl group, C₂-C₃₅ alkene group, or C₂-C₃₅ heteroalkene group; and
R¹⁷ and R¹⁸ are independently H, C₁-C₃₅ alkyl group, C₁-C₃₅ heteroalkyl group, C₂-C₃₅ alkene group, or C₂-C₃₅ heteroalkene group.

22. The elastomer of claim 16, wherein the citrate ester is a compound of formula (XIII) 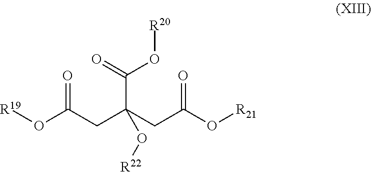
wherein
R¹⁹, R²⁰, R²¹, and R²² are independently H, C₁-C₃₅ alkyl group, C₁-C₃₅ heteroalkyl group, C₂-C₃₅ alkene group, or C₂-C₃₅ heteroalkene group.

23. The elastomer of claim 16, wherein the ether solvent is a compound of formula (XIV) 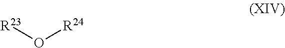
wherein
R²³ and R²⁴ are independently H, C₂-C₂₀ alkyl group, C₂-C₂₀ heteroalkyl group, C₂-C₂₀ alkene group, or C₂-C₂₀ heteroalkene group.

24. The elastomer of claim 23, wherein the ether solvent is selected from the group consisting of dicaprylyl ether, didecyl ether, panthenyl ethyl ether, dicetyl ether, dimyristyl ether, distearyl ether, distearyl ether, dilauryl ether, and combinations thereof.

25. The elastomer of claim 16, wherein the carbonate solvent is a compound of formula (XV) 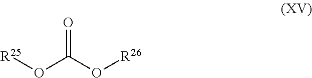
wherein
R²⁵ and R²⁶ are independently H, C₂-C₂₀ alkyl group, C₂-C₂₀ heteroalkyl group, C₂-C₂₀ alkene group, or C₂-C₂₀ heteroalkene group.

26. The elastomer of claim 16, wherein the hydrocarbon solvent is selected from the group consisting of farnesene, hydrogenated farnesene, coconut alkanes, coconut/palm kernel alkanes, C₉-C₁₂ alkane, C₁₀-C₁₃ alkane, C₁₂-C₁₇ alkane, C₁₃-C₁₄ alkane, C₁₃-C₁₅ alkane, C₁₄-C₁₇ alkane, C₁₄-C₁₉ alkane, C₁₄-C₂₀ alkane, C₁₄-C₂₂ alkane, C₁₅-C₁₉ alkane, C₂₁-C₂₈ alkane, C₁₇-C₂₃ alkane, C₉-C₁₂ isoalkane, C₉-C₁₃ isoalkane, C₉-C₁₄ isoalkane, C₉-C₁₆ isoalkane, C₁₀-C₁₁ isoalkane, C₁₀-C₁₂ isoalkane, C₁₀-C₁₃ isoalkane, C₁₁-C₁₂ isoalkane, C₁₁-C₁₃ isoalkane, C₁₁-C₁₄ isoalkane, C₁₂-C₁₄ isoalkane, C₁₂-C₁₅ isoalkane, C₁₂-C₂₀ isoalkane, C₁₃-C₁₄ isoalkane, C₁₃-C₁₆ isoalkane, C₁₄-C₁₆ isoalkane, C₁₅-C₁₉ isoalkane, diethylhexylcyclohexane, undecane, tridecane, tetradecane, pentadecane, hexadecane, octadecane, docosane, squalane, hydrogenated polyisobutene, polybutene, hydrogenated polydecene, hydrogenated didecene, mineral oil, liquidum, petrolatum, dodecane, isohexadecane, isododecane, isoeicosane, and combinations thereof.

27. A composition comprising the elastomer of claim 1.

28. The composition of claim 27, wherein the composition is a gel.

29. A gel produced by combining an elastomer of claim 1 with one or more solvents and processed by homogenization.

30. A personal care formulation comprising the gel of claim 29.

31. The personal care formulation of claim 30, wherein the personal care formulation is a personal care application selected from the group consisting of a deodorant, an antiperspirant, a skin cream, a facial cream, a hair shampoo, a hair conditioner, a mousse, a hair styling gel, a hair spray, a protective cream, a lipstick, a facial foundations, blushes, makeup, and mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.

32. A method of preparing an elastomer of claim 1 comprising reacting (i) at least one polycarboxylic acid, at least one polycarboxylic acid ester, or a combination thereof and (ii) at least one polyol in a first solvent thereby forming a crosslinking polymer structure.

33. The method of claim 32, wherein the method further comprises:
a) combining the crosslinking polymer structure with a second solvent thereby forming a swollen crosslinking polymer structure and
b) subjecting the swollen crosslinking polymer structure to shear force thereby forming a uniform polyester elastomer gel.
